(19) United States
(12) Patent Application Publication
(10) Pub. No.: US 2020/0305772 A1
(43) Pub. Date: Oct. 1, 2020

(54) BLOOD GLUCOSE MEASURING DEVICE, BLOOD GLUCOSE MEASURING SYSTEM, AND METHOD FOR MEASURING BLOOD GLUCOSE USING BLOOD GLUCOSE MEASURING DEVICE

(71) Applicant: Samsung Electronics Co., Ltd., Suwon-si, Gyeonggi-do (KR)

(72) Inventors: Young-jae OH, Suwon-si (KR); Hyoung-seon CHOI, Seoul (KR); Seong-je CHO, Suwon-si (KR); Seo-young YOON, Seoul (KR); Kyoung-jin MOON, Suwon-si (KR); Chul-ho CHO, Yongin-si (KR)

(21) Appl. No.: 16/753,954

(22) PCT Filed: Oct. 11, 2018
(86) PCT No.: PCT/KR2018/011912

§ 371 Date: Apr. 6, 2020

(30) Foreign Application Priority Data
Nov. 20, 2017 (KR) 10-2017-0155079

(51) Int. Cl. A61B 5/1495; A61B 5/145; A61B 5/00

(52) U.S. Cl. CPC A61B 5/1495; A61B 5/14532; A61B 5/0004; A61B 5/14503; A61B 2560/0223; A61B 5/742

(57) ABSTRACT

Disclosed are a blood glucose measuring device, a blood glucose measuring system, and a method for measuring blood glucose using the blood glucose measuring device. The present blood glucose measuring device comprises: a sensor for measuring blood glucose via a body fluid of a user; and a processor for obtaining error information of the sensor by comparing a first blood glucose level measured by the sensor, and a second blood glucose level measured via the blood of the user, at a first calibration interval during a preset time; calculating the time taken for the error range of the sensor to reach a preset threshold value, on the basis of the first calibration interval and the error information of the sensor; and setting the first calibration interval as a second calibration interval on the basis of the calculated time.

### TECHNICAL FIELD

This disclosure relates to a blood glucose measuring device and a blood glucose measuring method using the same. More particularly, the disclosure relates to a blood glucose measuring device and a method for measuring blood glucose using the same. The disclosure also relates to a blood glucose measuring system providing a blood glucose level of a user.

### BACKGROUND ART

Recently, patients suffering from chronic disease such as diabetes have gradually increased due to a variety of causes such as wrong eating habits, lack of exercise, stress, or the like.

In particular, in the case of chronic diseases such as diabetes, a patient needs to periodically measure the patient's own blood glucose and take appropriate action. For this purpose, various portable personal medical devices such as a blood glucose system, an insulin pump, or the like, have been recently developed.

There is a blood-collecting blood glucose system as one of medical devices for measuring blood glucose. In the case of the blood-gathering blood glucose system, the blood is collected directly through a wound which is generated by penetrating a needle under the skin, and the blood glucose is measured through the blood collected. However, this approach may cause a patient to feel pain in the course of blood collection. Accordingly, there has been a problem in that a patient is reluctant to have or avoid blood glucose measurement, and accordingly, the patient does not frequently check his/her blood glucose level, resulting in poor blood glucose control.

To overcome this limitation, recently, it has been developed a minimally invasive glucose sensor capable of continuously measuring blood glucose by being attached to the body of a patient. In the case of minimally invasive blood glucose systems, by penetrating the sensor to the skin and continuously measuring the glucose concentration in the body fluid, the user may be continuously provided with the blood glucose level. Thus, the patient may identify and control his or her blood glucose frequently.

However, in the case of the minimally invasive blood glucose system, the blood glucose is measured not by directly collecting blood. Therefore, the blood glucose level measured by the minimally invasive blood glucose system may be different from the actual blood glucose level, that is, the blood glucose level measured through the collected drawn. That is, an error may occur.

In order to address the problem, the minimally invasive blood glucose system provides a patient with calibrated blood glucose measured. However, in order to calculate the calibration value, blood glucose needs to be measured by directly collecting the blood of the patient. This is because the calibration value is calculated based on the difference between the blood glucose level measured from the blood and the blood glucose level measured by the minimally invasive blood glucose system.

In the related-art minimally invasive blood glucose system, the calibration value is calculated every default calibration interval. In general, a calibration interval set by a manufacturer is 12 hours.

In this example, when 12 hours have passed since the previous calculation of the calibration value, even though the blood glucose level calculated based on the calibration value by the minimally invasive blood glucose system and the actual blood glucose system is not that different, a patient needs to go through blood-gathering to calculate the calibration value of the minimally invasive blood glucose system and there is a problem that the patient may feel pain during the blood-collecting process.

On the contrary, before 12 hours from the previous calculation of the calibration value, even though there is a significant difference between the blood glucose level calculated by the minimally invasive blood glucose system based on the calibration value and the actual blood glucose level, the calibration value is calculated on 12-hour basis only and thus, there is a problem that accurate blood glucose level is not provided with the user.

Accordingly, there is a need for a minimally invasive blood glucose system that may adjust a calibration cycle to provide accurate blood glucose levels while minimizing pain to a patient.

The minimally invasive blood glucose system has a problem in that the accuracy of measurement is degraded over time. This is because the sensor of the minimally invasive blood glucose system continues to be inserted into the skin of a patient and thus is affected by various component materials present in the body fluid accordingly.

Nevertheless, the related-art minimally invasive blood glucose system only provides a patient with a blood glucose level calculated based on the calibration values calculated at the time corresponding to the calibration interval.

Accordingly, the related-art minimally invasive blood glucose system may only provide a blood glucose level only in the vicinity of a time point corresponding to a calibration interval, and thereafter may provide the blood glucose level with low accuracy as if the low value is an actual blood glucose level of the patent, making the patient not accurately manage blood glucose.

In the case of a noninvasive glucose sensor for measuring blood glucose level without inserting a sensor into the skin of a patient, there is a similar problem as the minimally invasive blood glucose system described above.

### DISCLOSURE

### Technical Problem

The disclosure provides a blood glucose measuring device, a blood glucose measuring system for adjusting a calibration interval of the blood glucose measuring device, providing an error range of a sensor along with the blood glucose level of a user, and a method for measuring blood glucose using the blood glucose measuring device.

### Technical Solution

A blood glucose measuring device according to an embodiment includes a sensor for measuring blood glucose via a body fluid of a user and a processor configured to obtain error information of the sensor by comparing a first blood glucose level measured by the sensor, and a second blood glucose level measured via the blood of the user, at a first calibration interval during a preset time, calculate a time taken for the error range of the sensor to reach a preset threshold value, on the basis of the first calibration interval and the error information of the sensor, and set the first calibration interval as a second calibration interval on the basis of the calculated time.

The processor may obtain first error information of the sensor by comparing a first blood glucose level measured by the sensor and a second blood glucose level measured via the blood of the user, at the first calibration time included in the preset time, obtain second error information of the sensor by comparing a first blood glucose level measured by the sensor and a second blood glucose level measured via the blood of the user at a second calibration time in which a time corresponding to the first calibration interval has passed from the first calibration time, and obtain error information of the sensor based on the first error information and the second error information.

The processor may obtain error information of the sensor by further considering a physical error range of the blood glucose measuring device itself included in each of the first error information and the second error information. The processor may, based on a time for the error range of the sensor to reach a preset threshold value being shorter than the first calibration interval, set the time to reach the preset threshold value as the second calibration interval, and based on the time to reach the preset threshold value being longer than the first calibration interval, and based on the time to reach the preset threshold value being in a preset correlation with the first calibration interval, set the first calibration interval as the second calibration interval.

The processor may, based on the time to reach the preset threshold value being longer than the first calibration internal and shorter than two times of the first calibration interval, set the second calibration interval to be identical with the first calibration interval, and based on the time to reach the preset threshold value being longer than the two times of the first calibration interval and shorter than three times of the first calibration interval, set the second calibration interval as two times of the first calibration interval.

The processor may, based on an error of the sensor being calibrated at the first calibration time, generate information on a blood glucose level including a blood glucose level measured by the sensor in a preset time interval unit from the first calibration time and a predicted error range of the sensor based on the error information of the sensor at the blood glucose measurement time.

The processor may provide an alarm to direct a user to calibrate an error of the sensor at a time corresponding to the second calibration interval.

According to an embodiment, a blood glucose measuring system including a blood glucose measuring device and a display device includes a blood glucose measuring device configured to obtain error information of a sensor of the blood measuring device by comparing a first blood glucose level measured via a body fluid of a user and a second blood glucose level measured via the blood of the user, at a first calibration interval during a preset time, based on the error of the sensor being calibrated at a first calibration time, measure a blood glucose level in a preset time interval unit from the first calibration time, predict an error range of a sensor based on the error information of the sensor at the measurement time of the blood glucose, and transmit, to the display device, information on a blood glucose level including the measured blood glucose level and the predicted error range of the sensor at the measurement time, and a display device configured to receive and display blood glucose information from the blood glucose measuring device.

The blood glucose measuring device may, based on the first calibration interval and the error information of the sensor, calculate a time taken for the error range of the sensor to reach a preset threshold value on the basis of the first calibration interval and the error information of the sensor, set the first calibration interval as a second calibration interval on the basis of the calculated time, and transmit information on the second calibration interval to the display device, wherein the display device is further configured to provide a user interface (UI) for guiding to calibrate an error of the sensor based on information on the second calibration interval.

The display device may, based on the predicted error range of the sensor at the measurement time corresponding to a preset threshold value, provide a preset visual feedback.

The display device may, based on a change amount of blood glucose measured by the sensor exceeding a preset threshold value, provide the UI by reflecting additional error information based on the change amount of blood glucose to the predicted error range of the sensor at the measurement time.

According to an embodiment, a method for measuring blood glucose using a blood glucose measuring device includes obtaining error information of a sensor of the blood measuring device by comparing a first blood glucose level measured via a body fluid of a user and a second blood glucose level measured via the blood of the user, at a first calibration interval during a preset time; calculating a time taken for the error range of the sensor to reach a preset threshold value, on the basis of the first calibration interval and the error information of the sensor; and setting the first calibration interval as a second calibration interval on the basis of the calculated time.

The obtaining the error information may include obtaining first error information of the sensor by comparing a first blood glucose level measured by the sensor and a second blood glucose level measured via the blood of the user, at a first calibration time included in the preset time, to calibrate the error of the sensor, obtaining second error information of the sensor by comparing a first blood glucose level measured by the sensor and a second blood glucose level measured via the blood of the user at a second calibration time in which a time corresponding to the first calibration interval has passed from the first calibration time, and obtaining error information of the sensor based on the first error information and the second error information.

The obtaining the error information may include obtaining error information of the sensor by further considering a physical error range of the blood glucose measuring device itself included in each of the first error information and the second error information.

The setting the second calibration interval may include, based on a time taken for the error range of the sensor to reach a preset threshold value being shorter than the first calibration interval, setting the time to reach the preset threshold value as the second calibration interval, and based on the time to reach the preset threshold value being longer than the first calibration interval, and based on the time to reach the preset threshold value being in a preset correlation with the first calibration interval, setting the first calibration interval as the second calibration interval.

The setting the second calibration interval may include, based on a time taken for the error range of the sensor to reach a preset threshold value being longer than the first calibration interval and shorter than two times of the first calibration interval, setting the second calibration interval to be identical with the first calibration interval, and based on the time to reach the preset threshold value being longer than the two times of the first calibration interval, and shorter than the three times of the first calibration interval, setting the second calibration interval as the two times of the first calibration interval.

The method of measuring the blood glucose may further include, based on an error of the sensor being calibrated at the first calibration time, generating information on a blood glucose level including a blood glucose level measured by the sensor in a preset time interval unit from the first calibration time and a predicted error range of the sensor based on the error information of the sensor at the blood glucose measurement time.

The measuring a blood glucose level may further include providing an alarm to direct a user to calibrate an error of the sensor at a time corresponding to the second calibration interval.

### Effect of Invention

According to various embodiments, a blood glucose measuring device capable of resetting a calibration interval is provided in consideration of an error degree of a sensor which differs by users, thereby minimizing inconvenience and pain of a user.

There is an effect of managing own blood glucose more accurately by providing an error range of a sensor predicted during blood glucose measuring along with a blood glucose level of a user.

### DESCRIPTION OF DRAWINGS

FIG. 1 is a diagram illustrating a blood glucose system according to an embodiment;

FIG. 2 is a diagram illustrating a blood glucose measuring device according to an embodiment;

FIG. 3A is a diagram illustrating a related-art blood glucose measuring device and a calibration interval of a blood glucose measuring device according to an embodiment;

FIG. 3B is a diagram illustrating a related-art blood glucose measuring device and a calibration interval of a blood glucose measuring device according to an embodiment;

FIG. 4A is a diagram illustrating blood glucose information provided by a related-art blood glucose measuring device;

FIG. 4B is a diagram illustrating blood glucose information provided by a blood glucose measuring system according to an embodiment;

FIG. 4C is a diagram illustrating blood glucose information provided by a blood glucose measuring system according to an embodiment;

FIG. 5 is another diagram illustrating blood glucose information provided by a blood glucose measuring system according to an embodiment;

FIG. 6 is another diagram illustrating blood glucose information provided by a blood glucose measuring system according to an embodiment;

FIG. 7A is a diagram illustrating providing a UI for directing calibration by a blood glucose measuring system according to an embodiment;

FIG. 7B is a diagram illustrating providing a UI for directing calibration by a blood glucose measuring system according to an embodiment;

FIG. 8 is a diagram illustrating a blood glucose measuring system for providing a UI reflecting additional error information if a change amount of the blood glucose level of a user exceeds a preset threshold value;

FIG. 9 is a detailed block diagram illustrating a blood glucose measuring device according to an embodiment;

FIG. 10 is a detailed block diagram illustrating a display device according to an embodiment; and

FIG. 11 is a flowchart illustrating an operation method of a blood glucose measuring device according to an embodiment.

### DETAILED DESCRIPTION OF EXEMPLARY EMBODIMENTS

Various modifications may be made to the embodiments of the disclosure, and there may be various types of embodiments. Accordingly, specific embodiments will be illustrated in drawings, and the embodiments will be described in detail in the detailed description. However, it should be noted that the various embodiments are not for limiting the scope of the disclosure to a specific embodiment, but they should be interpreted to include all modifications, equivalents or alternatives of the embodiments included in the ideas and the technical scopes disclosed herein. Meanwhile, in case it is determined that in describing embodiments, detailed description of related known technologies may unnecessarily confuse the gist of the disclosure, the detailed description will be omitted.

In addition, expressions “first”, “second”, or the like, used in the disclosure may indicate various components regardless of a sequence and/or importance of the components, may be used in order to distinguish one component from the other components, and do not limit the corresponding components.

The terminology used in this application is for the purpose of describing particular embodiments only and is not intended to limit the scope of the claims. A singular expression includes a plural expression, unless otherwise specified. It is to be understood that the terms such as “comprise” or “include” are used herein to designate a presence of a characteristic, number, step, operation, element, component, or a combination thereof, and not to preclude a presence or a possibility of adding one or more of other characteristics, numbers, steps, operations, elements, components or a combination thereof.

The term such as “module,” “unit,” “part”, and so on may be used to refer to an element that performs at least one function or operation, and such element may be implemented as hardware or software, or a combination of hardware and software. Further, except for when each of a plurality of “modules”, “units”, “parts”, and the like needs to be realized in an individual hardware, the components may be integrated in at least one module or chip and be realized in at least one processor.

Hereinafter, various embodiments of the disclosure will be described in greater detail with reference to the attached drawings.

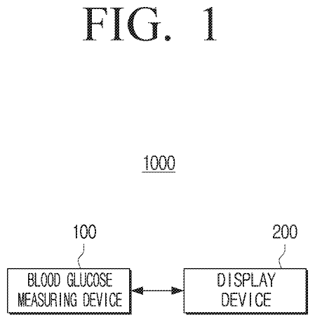

FIG. 1 is a diagram illustrating a blood glucose system according to an embodiment.

Referring to FIG. 1, a blood glucose measuring system 1000 according to an embodiment may include a blood glucose measuring device 100 and a display device 200.

The blood glucose measuring device 100 may measure blood glucose of a user. Here, blood glucose may mean glucose concentration in the blood of a user, and the blood glucose measuring device 100 may measure blood glucose level through glucose in blood collected via blood collection.

The blood glucose measuring device 100 may also measure blood glucose via the body fluid of the user. Here, blood glucose may refer to glucose concentration in a user's body fluid, and the blood glucose measuring device 100 may measure blood glucose level through glucose that diffuses from blood and is present in the user's body fluid. Here, the body fluid may include, but is not limited to, interstitial fluid, sweat, tears, saliva, or the like.

The blood glucose measuring device 100 may be a device of a type attached to the user's skin. For example, the blood glucose measuring device 100 may be implemented with a minimally invasive blood glucose measuring device or a non-invasive blood glucose measuring device in various forms, such as a patch form attached to the skin, a watch form attached to the wrist, or the like.

The blood glucose measuring device 100 may measure blood glucose level via the body fluid of the user in a preset time unit. Here, the preset time unit may be set in various time units such as one minute, ten minutes, one hour, or the like.

The blood glucose measuring device 100 may then calibrate the measured blood glucose level. Specifically, the blood glucose measuring device 100 may perform calibration based on a difference value between the blood glucose level measured from the blood and the blood glucose level measured via the body fluid.

Unless otherwise mentioned, the blood glucose level measured by the blood glucose measuring device 100 refers to the blood glucose level measured through the body fluid of the user.

The blood glucose measuring device 100 may transmit blood glucose information to the display device 200. Here, the blood glucose information may include blood glucose level calculated based on the blood glucose level measured by the blood glucose measuring device 100 or the blood glucose level calculated based on the calibration value in a preset time interval.

The blood glucose information may further include information about an error range of the blood glucose measuring device 100 that is predicted at the time of measuring blood glucose level. Here, the information on the error range may be the difference in glucose concentration in the blood predicted at the time the blood glucose measuring device 100 measures blood glucose and the concentration of glucose in the body fluid. For example, if the user's blood glucose level measured by the blood glucose measuring device 100 at the first time point is A, and the error range of the blood glucose measuring device 100 predicted at the first time point is ±10%, the blood glucose measuring device 100 may transmit information about the blood glucose level A and information about the expected error range ±10% to the display device 200.

The error of the blood glucose measuring device 100 may be generated by a variety of causes. Specifically, glucose in blood takes a certain time to completely diffuse into the body fluid in the user's skin. Accordingly, there may be a difference between the glucose concentration of the blood and the glucose concentration of the body fluid, and thus, the blood glucose level measured by the blood glucose measuring device 100 via the body fluid or the blood glucose level calculated by the blood glucose measuring device 100 may differ from the blood glucose level measured via the actual blood. In addition, an error may occur in the blood glucose measuring device 100 by the influence of the various component materials present in the body fluid other than glucose.

The error of the blood glucose measuring device 100 may vary by users, because the internal environment is different for each user. Specifically, the time taken for the glucose in the blood to completely diffuse into the body fluid in the blood of the user may be different by users as body characteristics are different by users, and the component materials present in the body fluid other than glucose may be different by users, as medical history, insulin injection status, or the like, may be different by users.

The blood glucose measuring device 100 itself may have own error range.

The display device 200 may display various images. The display device 200 may display information on blood glucose received from the blood glucose measuring device 100.

The user may recognize the blood glucose level measured by the blood glucose measuring device 100 or calculated blood glucose level, or an error range of the blood glucose measuring device 100.

Thus, there is an effect that the user may control own health state in consideration of the blood glucose level measured by the blood glucose measuring device 100 or the calculated blood glucose level together.

Specifically, the actual blood glucose may be within a hyperglycemic to hypoglycemic range, in that, if the blood glucose levels measured or calculated by the blood glucose measuring device 100 are within a normal numerical range, as there may be an error.

In this case, if only the blood glucose level measured or calculated by the blood glucose measuring device 100 is displayed, as in the related-art blood glucose measuring device, the user may consider his/her health condition in a normal state and may neglect health care.

The display device 200 according to an embodiment may prevent the above problem by displaying an error range of the blood glucose measuring device 100 as well, based on the blood glucose information received from the blood glucose measuring device.

For example, if the blood glucose level measured or calculated by the blood glucose measuring device 100 is A in the normal range and the error range of the blood glucose measuring device 100 is ±10%, the display device 200 receives blood glucose information including such information from the blood glucose measuring device 100. The display device 200 displays the blood glucose level A and the error range ±10% of the blood glucose measuring device 100 together and thus, the user may recognize that the actual blood glucose may be a value of ±10% of the blood glucose A, but not A, which is a blood glucose level measured or calculated by the blood glucose measuring device 100.

If the value to which the error range of ±10% is applied to the blood glucose level A is included in the hyperglycemic range to hypoglycemic range, the user may recognize that his or her current blood glucose may be included in the hyperglycemic or hypoglycemic range, and accordingly, the user may strictly care the blood glucose state by checking blood glucose once again, managing meal plans, visiting a hospital, or the like.

It has been described that the blood glucose measuring device 100 and the display device 200 exist as separate devices, but the embodiment is not limited thereto. For example, the blood glucose measuring device 100 and the display device 200 may be integrated into a single device. In this case, the blood glucose measuring device 100 may provide a user with blood glucose information by including a display (not shown) and implementing the same operation as the display device 200 described above.

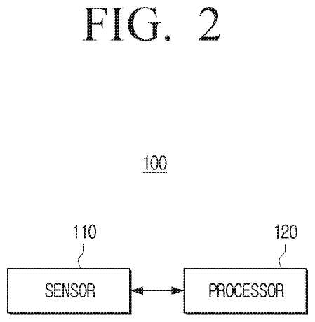

FIG. 2 is a diagram illustrating a blood glucose measuring device according to an embodiment.

Referring to FIG. 2, the blood glucose measuring device 100 according to an embodiment includes the sensor 110 and the processor 120.

The sensor 110 may measure blood glucose of a user. The blood glucose may refer to glucose concentration in blood of a user, and the sensor 110 may measure blood glucose through glucose in blood collected by blood collection.

The sensor 110 may measure blood glucose through the body fluid of the user. Specifically, the sensor 110 may measure blood glucose through glucose that diffuses from the blood and is present in the user's body fluid. Here, the body fluid may include, but is not limited to, interstitial fluid, sweat, tears, saliva, or the like.

The processor 120 controls overall operations of the blood glucose measuring device 100.

As described above, the blood glucose measuring device 100 may generate an error by a variety of causes. Here, the error of the blood glucose measuring device 100 may specifically refer to the error of the sensor 110. Accordingly, to provide the user with an accurate blood glucose level, the processor 120 may calculate a calibration value by comparing the blood glucose level measured by the sensor 110 with the blood glucose level measured via the blood, and then provide the user with a blood glucose level that is obtained by calibrating the blood glucose level measured by the sensor 110 based on the calculated calibration value

Hereinbelow, for convenience, it will be described that the processor 120 may, for example, calculate a first calibration value at the first calibration time point and calculate a second calibration value at the second calibration time point from which a preset time corresponding to a calibration interval has passed.

The processor 120 may calculate a calibration value by comparing the blood glucose level measured by the sensor 110 and the blood glucose level measured via the blood every preset calibration interval.

For example, the processor 120 may calculate a calibration value by comparing the blood glucose level measured by the sensor 110 and the blood glucose level measured via the blood at the first calibration time point.

The processor 120 may calibrate blood glucose level measured by the sensor 110 and provide the same to a user based on a calibration value calculated at the first calibration time point from the first calibration time point before the second calibration time point.

The processor 120 may calculate the calibration value by comparing the blood glucose level measured by the blood glucose measuring device 100 with the blood glucose level measured via the blood at the second calibration time point, and from the second calibration time point before the third calibration time point, may calibrate the blood glucose level measured by the sensor 110 and provide the user with the blood glucose level based on the calibration value calculated at the second calibration time point, in the same manner as the above method.

The processor 120 may obtain the error information of the sensor 110 by comparing the blood glucose level measured by the sensor 110 and the blood glucose level measured via the blood of the user in a preset calibration interval for a preset time.

That is, in the embodiment described above, the processor 120 may obtain the error information of the sensor 110 based on differences in blood glucose levels measured by the sensor 110 at each calibration time point and blood glucose levels measured via blood. The preset time may have been set when the product is manufactured, and may be set differently by the user. For example, the preset time may be, for example, one week, one month, or the like.

For example, if the preset time may be the time between the first calibration time point and the second calibration time point, and the blood glucose level measured by the blood glucose measuring device 100 is 1.10×A, the blood glucose level measured via the blood is A at the first calibration time point, and the blood glucose level measured by the blood measuring device 100 is 1.10×A, and the blood glucose level measured via the blood is A at the second calibration time, in the same manner, the processor 120 may obtain the error information of 10% during the time interval between the first calibration time point and the second calibration time point.

In the embodiment described above, it has been described that the sensor error information at the first calibration time point and the second calibration time point is the same, as an example, but depending on cases, error information of a sensor may be different for each calibration time point.

In this example, the processor 120 may obtain the error information of the sensor 110 based on an average value of the sensor error information at the first calibration time point and an average value of the sensor error information at the second calibration time point.

The processor 120 may compare the blood glucose level measured by the sensor 110 and the blood glucose level measured via the blood of the user at the first calibration time point, and then obtain the difference value as the first error information.

After calibrating the error of the sensor 110, the processor 120 may compare the blood glucose value measured by the sensor 110 and the blood glucose value measured via the blood of the user at a second calibration time corresponding to a subsequent calibration interval of the first calibration time point, and then obtain the difference value as the second error information.

The processor 120 may obtain the error information of the sensor 110 based on the first and second error information.

For example, if the first error information of the first calibration time point is 10% and the second error information of the second calibration time point is 12%, the processor 120 may obtain the average value 11% as the error information of the sensor 110 using the first and second error information.

The processor 120 may calculate a time when the error degree of the sensor 110 reaches a preset threshold value based on the preset calibration interval and the obtained error information of the sensor 110. Here, the preset threshold value may be a value set when manufacturing a product, and may be a value set by a user.

The preset threshold value may refer to a value indicating an accuracy of the sensor 110. For example, when the preset threshold value is 80%, the accuracy of the sensor 110 may refer to 80%.

In the above-described embodiment, if the preset calibration interval is 12 hours, the processor 120 may confirm that the error increase rate of 10%/12h occurs during the time interval between the first and second calibration time points. That is, the processor 120 may confirm that the error of the sensor 110 increases by 10% every 12 hours.

If the preset threshold value is set to 80%, that is, if the error degree of the sensor 110 is set to be tolerable up to 20%, the processor 120 may calculate the time at which the error degree of the sensor 110 reaches a preset threshold value as 24 hours through the operation of 10(%): 12 (h)=20(%): x (h).

The processor 120 may change a preset calibration interval based on the calculated time. Here, if the preset calibration interval is referred to as the first calibration interval in the embodiment described above, since the time for reaching the preset threshold value is 24 hours, the first calibration interval, which has been 12 hours, may be changed to the second calibration interval of 24 hours.

The calibration interval changed as specified above may be different by users. As described above, the internal environment of each user is different and thus, if users use the same sensor 110, the error of the sensor 110 may be obtained in a different manner.

In the above-described embodiment, it has been described that the calibration interval becomes longer than the preset calibration interval, but the calibration interval may be changed to be shorter depending on users. For example, if the error increase rate of the sensor 100 is 15%/12 h, and the preset threshold value is 87.5%, that is, the error degree of the sensor 110 is set to be tolerable up to 12.5%, the processor 120 may calculate the time to reach the threshold value by 10 hours through the operation. In this example, the processor 120 may change the calibration interval to 10 hours which is a calibration interval shorter than 12 hours that is a preset calibration interval.

In the embodiment as described above, it has been described that the physical error range of the sensor 110 itself has been excluded. However, an actual sensor 110 may have its own error range that is not relevant with the body characteristic of a user.

The processor 120 may change the preset calibration interval by further considering the physical error range of the blood glucose measuring device 110, that is, the sensor 110 itself.

For example, it will be described that the physical error range of the sensor is ±5%.

In this example, if the difference between the first error information of the first calibration time point, that is, the blood glucose level measured by the sensor 110 and the blood glucose level measured via the blood of the user is 7%, the processor 120 may confirm that the physical error range of the sensor 110 is ±5%, and identify the remaining 2% as the error of the sensor 110 attributable to the user's characteristics.

The processor 120 provides a user with blood glucose in which 7% error is calibrated from the blood glucose level measured by the sensor 110, after the first calibration time before the second calibration time.

If the difference between the second error information of the second calibration time point, that is, the blood glucose level measured by the sensor 110 and the blood glucose level measured via the blood of the user is 7% in the same manner as the first calibration time point, the processor 120 may confirm that the physical error range of the sensor 110 is ±5%, and identify the remaining 2% as the error of the sensor 110 attributable to the user's characteristics.

The processor 120 may obtain the error information that the physical error range of the sensor 110 is ±5%, and the error of the sensor based on the user's characteristic is 2%.

In the embodiment as described above, it has been described that the error of the sensor 110 is the same at the first calibration time and the second calibration time, but the error of the sensor 110 may be different for each calibration time.

The processor 120 may obtain the error information of the sensor 110 based on an average value of the error information at the first calibration time and the second calibration time.

For example, if the error at the first calibration time is 6%, the processor 120 may confirm that the physical error range of the sensor 110 is ±5% and that the error attributable to the user's characteristic is 1%.

If the error in the second calibration time is 8%, the processor 120 may confirm that the physical error range of the sensor 110 is ±5%, and confirm that the error attributable to the user characteristic is 3%. Accordingly, the processor 120 may confirm that the error information of the sensor 110 is ±5%, and the error attributable to the user characteristic is 2% which is the average value.

The processor 120 may calculate a time taken for the error range of the sensor 110 to reach a preset threshold value based on the first and second error information and the physical error range of the sensor 110 itself.

The error of the sensor 110 itself may have a fixed value in that it is the physical error range. That is, the error is not increased over time, but it is possible to maintain a constant value. On the contrary, errors that occur due to user characteristics may be an error that continuously changes due to problems such as contact of the sensor 110 with the various component materials present in the user body fluid. That is, the error of the sensor attributable to the user characteristics may be increasingly larger over time.

If the physical error range of the sensor 110 itself is ±5%, the error attributable to the user's characteristic is 2%, and the preset calibration interval is 12 hours, the processor 120 may calculate a time taken for the error degree of the sensor 110 to reach a preset threshold value based on the physical error range ±5% of the sensor 110 and the error increase rate 2%/12 h attributable to the user's characteristic.

The processor 120 may confirm that the error range of the sensor 110 after 12 hours as ±7% based on the physical error range ±5% of the sensor 110 itself and the error rate 2%/12 h attributable to the user's characteristics, and may confirm that the error range of the sensor 110 after 24 hours as ±9% based on the physical error range ±5% of the sensor 110 itself and the error rate 4%/24 h attributable to the user's characteristics. Consequently, the processor 120 may calculate, via the operation of 2(%): 12 (h)=5(%): x (h), the time at which the error degree of the sensor 110 reaches a preset threshold value as 30 hours.

The processor 120 may change the preset calibration interval to 30 hours.

It has been described that the calibration interval gets longer than a preset calibration interval, but the calibration interval may be changed to be shorter depending on users, as described above.

If the time taken to reach the preset threshold value is longer than the preset first calibration interval, the processor 120 may control the blood glucose measuring device 100 to change the calibration interval only when the time taken to reach the preset threshold value is in a preset correlation with the first calibration interval.

If the time to reach the preset threshold value is longer than the first calibration interval and shorter than two times of the first calibration interval, the processor 120 may maintain the first calibration interval, and if the time to reach the preset threshold value is longer than the two times of the first calibration interval and shorter than the three times of the first calibration interval, the processor 120 may change the calibration interval to two times of the first calibration interval.

As for a blood glucose measuring device, it is general that the calibration interval is 12 hours. However, blood through blood collection is required for calculating a calibration value, but if the calibration interval is not to set in a 12-hour unit, calibration may be performed at midnight or dawn, and may hinder a user to have a sound sleep, therefore it is not desirable.

Accordingly, if the preset calibration interval is 12 hours, when the time to reach the preset threshold value is calculated as 14 hours, the processor 120 may maintain the calibration interval as 12 hours and when the time to reach the preset threshold value is calculated as 25 hours, the processor 120 may change the calibration interval to 24 hours.

If the time to reach the preset threshold value is shorter than the preset first calibration interval, the processor 120 may change the time to reach the preset threshold value into the second calibration interval without considering the preset correlation relationship described above. This is because, unlike the case where the error degree of the sensor 110 is not large and the calibration time is delayed, the error degree of the sensor 110 is increased, and if the calibration interval of 12 hours is maintained, accurate blood glucose may not be provided by the error.

The embodiment is not necessarily limited thereto and even if the time to reach a preset threshold value is shorter than the first calibration interval, the preset first calibration interval may be set to be maintained.

The processor 120 may provide a notification to make a user calibrate an error of the sensor 110 at the time corresponding to the calibration interval.

Here, the notification may be a variety of types of notifications, such as visual, auditory, tactile feedback, or the like. For example, if the blood glucose measuring device 100 includes a display (not shown), the processor 120 may display guide information on the display (not shown) that directs to calibration when the calibration time has been reached, and if the blood glucose measuring device 100 includes a speaker (not shown), the processor 120 may output audio through a speaker (not shown) to direct the calibration when the calibration time has been reached. The processor 120 may induce calibration to the user by controlling the blood glucose measuring device 100 to vibrate, if the calibration time is reached.

It has been described that the preset calibration interval is changed once, but the blood glucose measuring device 100 according to an embodiment may continuously change the calibration interval by a preset time unit through the above-described method. The preset time unit may be set at the time of manufacturing of the product, or may be set by the user. For example, the preset time unit may be one week, one month, or the like.

If the first calibration interval is changed to the second calibration interval, the processor 120 may obtain the error information of the sensor 110 every second calibration interval, from the time when the first calibration interval is changed to the second calibration interval.

When a preset time unit, such as one month, has elapsed from the time when the first calibration interval has been changed to the second calibration interval, the processor 120 may calculate a time when the error degree of the sensor 110 reaches a preset threshold value based on the obtained error information, and change the second calibration interval to a third calibration interval based on the calculated time. In the same manner, the processor 120 may continuously change the calibration interval of the blood glucose measuring device 100 every preset time unit.

As described above, the blood glucose measuring device 100 according to an embodiment may continuously monitor the error of the sensor 110, and re-calibrate the calibration interval when the re-calibration of the calibration interval is necessary. Accordingly, the blood glucose measuring device 100 may continuously provide an accurate blood glucose level to a user, and may minimize the pain of a user by requiring blood collection only when calibration is necessary.

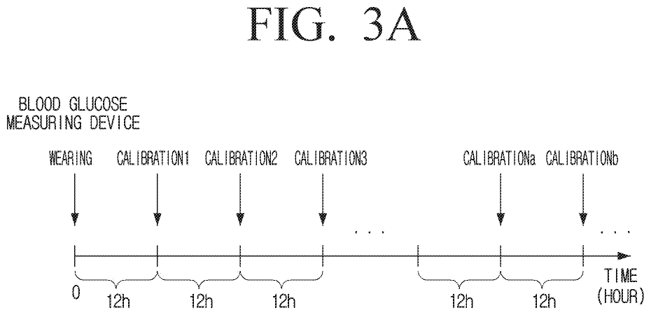

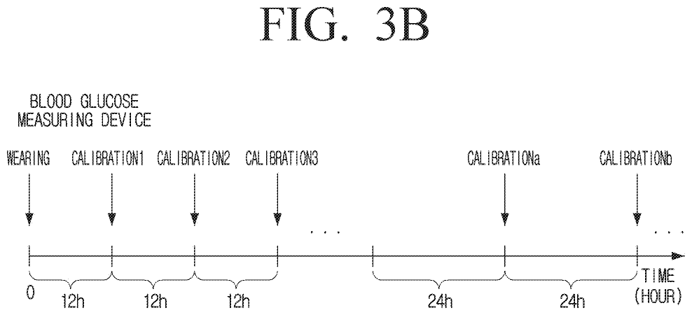

FIGS. 3A and 3B are diagrams illustrating a related-art blood glucose measuring device and a calibration interval of a blood glucose measuring device according to an embodiment.

Referring to FIG. 3A, a related-art blood glucose meter is calibrated with a preset calibration interval. That is, although the degree of error of the sensor 110 varies according to the physical characteristics of the user, the calibration is performed with a default calibration interval (for example, 12 hours) without considering this.

Accordingly, even though the error degree of the sensor 110 is not large actually, the related-art blood glucose measuring device may perform calibration through blood collection, causing inconvenience to a user.

In contrast, according to an embodiment, the blood glucose measuring device 100 may calculate a time for reaching a preset threshold value based on the error information of the sensor 110, and set the calculated time as a calibration interval.

For example, referring to FIG. 3B, the processor 120 may calculate the calibration value for calibrating the sensor 110 in 12-hour unit that is a preset first calibration interval, and obtain the error information of the sensor 110 at the time corresponding to the calibration interval.

Based on the error information of the sensor 110, if the time taken for the error degree of the sensor 110 to reach the preset threshold value is calculated as 24 hours, the processor 120 may change the calibration interval of the blood glucose measuring device 100 to a second calibration interval. That is, it may be changed from 12 hours to 24 hours.

In other words, unlike the related-art blood glucose measuring device which performs calculation only with a default calibration interval even if the error of the sensor 110 is not large, the calibration interval is increased if the error of the sensor 110 is not large, thereby minimizing the inconvenience to the user due to the blood collection.

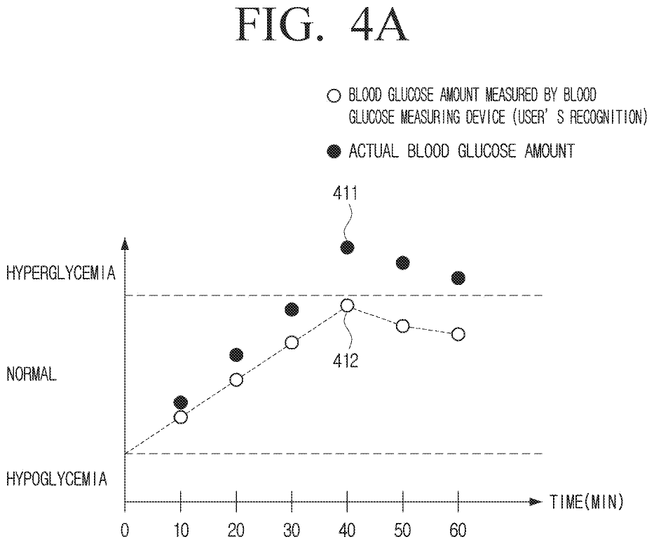

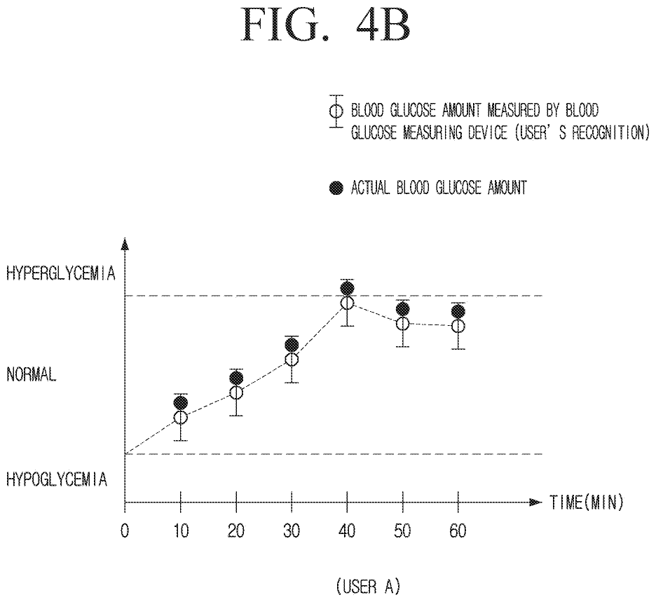

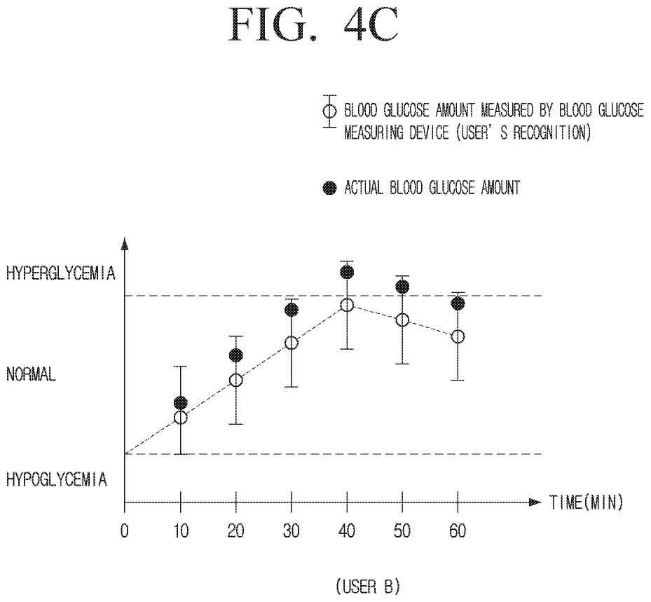

FIGS. 4A and 4B are diagrams illustrating blood glucose information provided by a related-art blood glucose measuring device.

The blood glucose measuring device 100 and the display device 200 will be described as separate devices, but as described above, the blood glucose measuring device 100 and the display device 200 may be integrated into a single device. In this case, the blood glucose measuring device 100 may further include a display (not shown).

Referring to FIG. 4A, a related-art blood glucose measuring device provided only the blood glucose measured by the blood glucose measuring device. However, an error may occur in the sensor of the blood glucose measuring device, so that the actual blood glucose value 411 is included within the hyperglycemic range, and the blood glucose value 412 provided by the blood glucose measuring device may be included within the normal range. As a result, a user does not properly recognize the hyperglycemia, and thus the user may not properly manage the blood glucose. The same is applied for hypoglycemia.

However, the blood glucose measuring device 100 according to an embodiment may have an effect to solve this problem by providing blood sugar information including the error range of the sensor 110 together with the blood glucose level measured by the sensor 110 to the user.

Referring to FIG. 4, the display device 200 may display not only blood glucose measured by the sensor 110 but also display the error range of the sensor 110 as well based on the error information of the sensor

For example, if the error range of the sensor 110 is ±10%, the display device 200 may display a UI reflecting an error range ±10% to the blood glucose value measured by the blood glucose measuring device 100.

Accordingly, the user may recognize that the blood glucose value measured by the sensor 110 in the 40-minute interval is within the normal numerical range, but current glucose level may correspond to hyperglycemia when the error range of the sensor 110 is considered together. In addition, the user may manage the blood glucose level through meal control, medicine dosing and the like before reaching the dangerous blood glucose level.

As described above, the error information of the sensor 110 may be different in that the internal body environment is different for each user. Thus, even when using the same blood glucose measuring device 100, the processor 120 may obtain the error information of the sensor 110 differently for each user, and the error range of the sensor 110 displayed on the display device 200 may be different by users.

For example, referring to FIGS. 4B and 4C, even if the same the blood glucose measuring device 100 is used, the display device 200 may display different error ranges, respectively, by receiving different error information from the blood glucose measuring device 100 depending on whether the user is user A or user B.

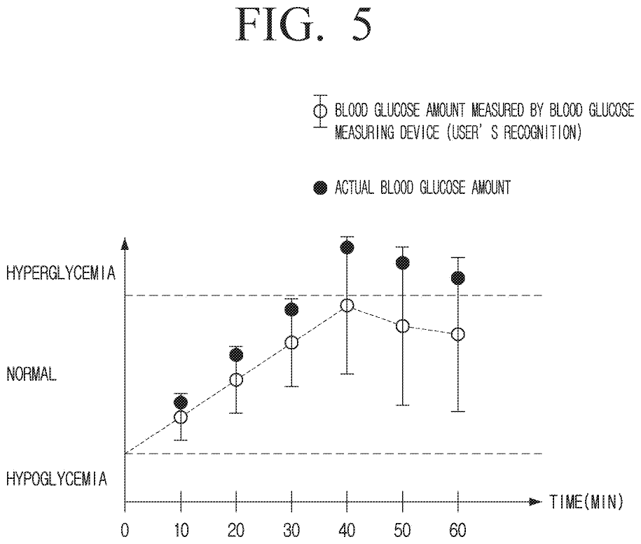

FIG. 5 is another diagram illustrating blood glucose information provided by a blood glucose measuring system according to an embodiment.

When the error of the sensor 110 is calibrated at the first calibration interval time point, the blood glucose measuring device 100 may measure blood glucose in units of a preset time interval from the first calibration time point, and predict an error range of the sensor based on the error information of the sensor at the time of measuring blood glucose.

For example, if the error increase rate of the sensor 110 is 10%/12 h, and the sensor 110 measures blood glucose in 10 minutes after calibration, the blood glucose measuring device 100 may predict the error range of the sensor 110 based on an error rate of 0.14%/10 min. That is, the blood glucose level measured by the sensor 110 when 10 minutes after calibration may have an error range of ±0.14%, and when 20 minutes are reached, the blood glucose level measured by the sensor 110 may be predicted to have a range of ±0.28%

The processor 120 may transmit to the display device 200 information including blood glucose information, blood glucose level measured by the sensor 110, and a predicted error range of the sensor at the measurement time.

Accordingly, the display device 200 may provide the UI including the blood glucose level measured in a predetermined time interval unit and the error range of the sensor predicted at the time of measurement based on the blood glucose information received from the blood glucose measuring device 100.

For example, referring to FIG. 5, the display device 100 may display a UI applying the error range of ±0.14% to the blood glucose level measured by the blood glucose measuring device 100 at a time corresponding to 10 minutes, and may display a UI applying the error range of ±0.28% to the blood glucose level measured by the blood glucose measuring device 100 at a time point corresponding to 20 minutes.

Accordingly, in the case of FIG. 5, the user may recognize that the blood glucose value measured by the sensor 110 at a time point corresponding to 40 minutes, 50 minutes, and 60 minutes is within the normal numerical range, but its current blood glucose level may correspond to hyperglycemia when the error range of the sensor 110 is considered together. The user may manage the blood glucose level through meal management, medicine dosing and the like, before reaching the dangerous blood glucose level.

As shown in FIG. 5, the display device 200 may provide a UI indicating that the error of the sensor 110 is gradually increasing after the first calibration time based on the received blood glucose information. Accordingly, the user may recognize that the error degree of the sensor 110 is gradually increasing, and in addition, it is possible to recognize that the calibration time point is nearer. Accordingly, the preparation for calibration may be performed in advance.

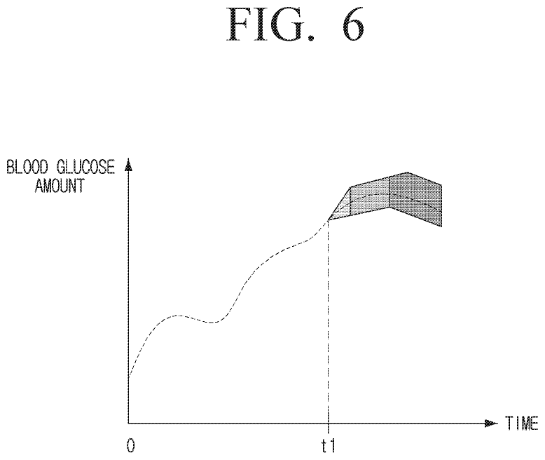

FIG. 6 is another diagram illustrating blood glucose information provided by a blood glucose measuring system according to an embodiment.

Based on the blood glucose information received from the blood glucose measuring device 100, if the sensor 110 confirms that the predicted error range of the sensor reaches a preset threshold value at the time when the blood glucose is measured by the sensor 110, the display device 200 may provide a preset visual feedback after the corresponding time point. For this purpose, the blood glucose information received by the display device 200 may further include information on a preset threshold value.

Here, the preset threshold value may be a value set in manufacturing a product, and may be a value set by a user. For example, if the preset threshold value is set to 95%, the display device 200 may provide a preset visual feedback from the point when the error of the sensor 110 is greater than or equal to 5%.

The visual feedback may refer to displaying color, brightness, or the like, differently. For example, when the predicted error range of the sensor 110 reaches a preset threshold value, the display device may display an error of the sensor 110 differently after the corresponding point or display by adjusting brightness.

The display device 200 may provide a visual feedback as a polygonal shape connecting a highest point with a lowest point of each error value of the sensor 110.

In addition, as shown in FIG. 6, when the predicted error of the sensor 110 reaches a preset threshold value, the display device 200 may gradually raise brightness inside the polygon by displaying each error value of the sensor 110 in the form of a polygon connecting the highest point and the lowest point.

Alternatively, the display device 200 may provide visual feedback by various methods. For example, in the UI as shown in FIG. 5, the display device 200 may differentiate the shape or color of a bar when the error range of the sensor 110 has reached a preset threshold value.

Accordingly, a user may receive a feedback that the error range of the sensor 110 is at a dangerous level and recognize necessity of calibration.

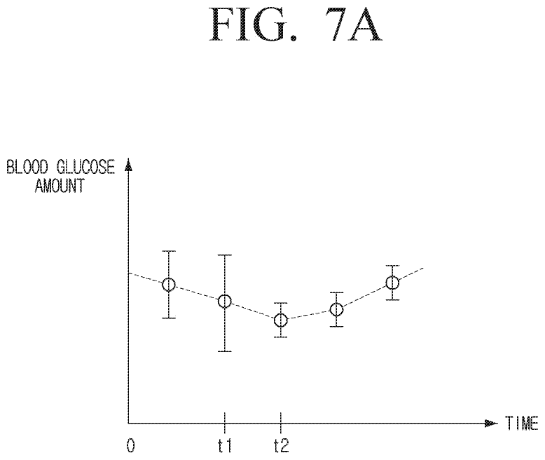

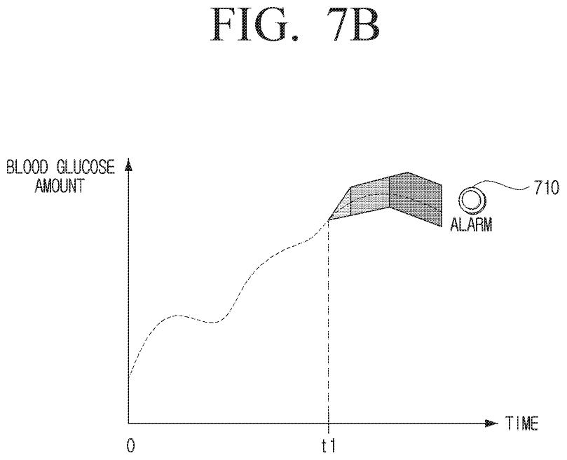

FIGS. 7A and 7B are diagrams illustrating providing a UI for directing calibration by a blood glucose measuring system according to an embodiment.

The display device 200 may receive information about the calibration interval of the blood glucose measuring device 100 from the blood glucose measuring device 100. The display device 200 may provide a UI for guiding to calibrate an error of the sensor 110 at a point where calibration of the blood glucose measuring device 100 is necessary based on the information about the calibration interval.

Referring to FIG. 7A, if it is identified that t1 is a time point when calibration is necessary, based on the calibration interval of the blood glucose measuring device 100, the display device 200 may display a text (not shown) that calibration is necessary at t1 time point.

As shown in FIG. 7A, when the user calibrates the blood glucose measuring device 100 at t1 time, the display device 200 may display a reduced error of the sensor 110 according to the calibration from the time t2, which is the time point for measuring blood glucose after t1. Accordingly, the user may identify that the sensor 110 has been properly calibrated.

According to an embodiment, in FIG. 7A, if the calibration interval of the blood glucose measuring device 100 is changed and the blood glucose measuring device 100 performs calibration with the first calibration interval before the time t1, and performs calibration with the second calibration interval after the time t1, the display device 200 may display a text (not shown) indicating that the calibration interval has been changed.

Referring to FIG. 7B, if it is identified that the time when calibration is necessary is reached, the display device 200 may display a notification UI 710 indicating that calibration is necessary.

In the above-described embodiment, it has been described, as an example, that the visual feedback is provided at the time required for calibration of the blood glucose measuring device 100, but the embodiment is not limited thereto. The display device 200 may provide haptic feedback, as well as direct the user to calibrate the blood glucose measuring device 100 via various feedback, such as auditory feedback.

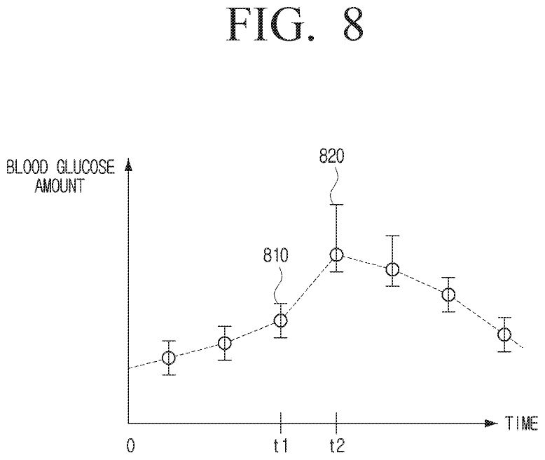

FIG. 8 is a diagram illustrating a blood glucose measuring system for providing a UI reflecting additional error information if a change amount of the blood glucose level of a user exceeds a preset threshold value.

The display device 200 may provide a UI reflecting the additional error information when the change amount of blood glucose measured by the blood glucose measuring device 100 exceeds a preset threshold value. In general, when the amount of change in blood glucose measured by the blood glucose measuring device is large, the degree of error may be greater than if the amount of change in blood glucose is small. This is because the glucose in the blood takes some time until it completely diffuses into the body fluid in the user's skin.

Accordingly, when a change amount of blood glucose measured by the sensor 110 is large, a UI reflecting the above needs to be provided. The display device 200 may provide a UI reflecting additional error information based on the change amount of blood glucose to the predicted error information of the sensor at the time of blood measuring time point.

For example, as shown in FIG. 8, if the measured blood glucose value 720 measured at second time point is greater than the blood glucose value 710 measured by the sensor 110 at the first time point by a preset threshold value or more, the display device 200 may display the UI reflecting the additional error information in the error information of the sensor 110. In the case of FIG. 8, the measured blood glucose level becomes higher by a preset threshold value or more, and in this case, the display device 200 may provide a UI that sets the upper limit of the error of the sensor to be higher.

The additional error information may be pre-stored in the display device 200 based on the degree of change of blood glucose. Alternatively, the display device 200 may further receive information regarding additional error information from the blood glucose measuring device 100. Alternatively, the display device 200 may receive additional error information based on the degree of change of blood glucose from an external device, such as a server.

In FIG. 8, it has been described that the blood glucose level rapidly increases, but in the case where the blood glucose level measured by the sensor 110 is rapidly decreased, the display device 200 may provide a UI reflecting the additional error information in a similar manner. In this case, the display device 200 may provide a UI that sets a lower limit range of the error to be higher.

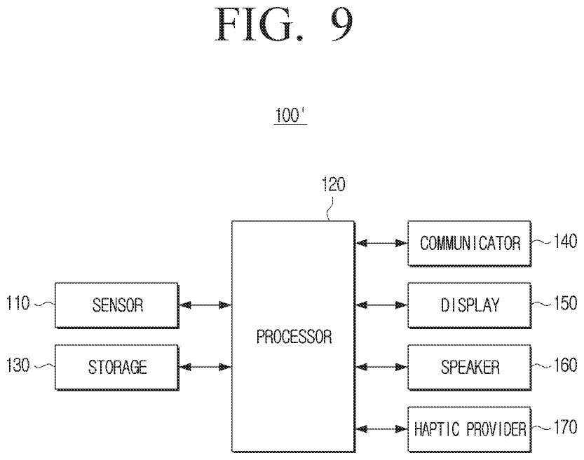

FIG. 9 is a detailed block diagram illustrating a blood glucose measuring device according to an embodiment. Hereinbelow, a part overlapping with the described part will not be described.

Referring to FIG. 9, a blood glucose measuring device 100′ according to an embodiment may include the sensor 110, the processor 120, a storage 130, a communicator 140, a display 150, a speaker 160, and a haptic provider 170.

The storage 130 may store an instruction or data related to a component of the blood glucose measuring device 100′ and the operating system (OS) for controlling overall operations of the component of the blood glucose measuring device 100′.

Accordingly, the processor 120 may control multiple hardware or software components of the blood glucose measuring device 100′ using various instructions or data stored in the storage 130, load instructions or data received from at least one of the other components into the volatile memory, and store the various data in the non-volatile memory. The storage 130 may store differences in the glucose concentration of the blood and the glucose concentration of the body fluid at the point of calibration to calculate a calibration value.

The communicator 140 may communicate with the display device 200 to transmit and receive various data. In particular, the communicator 140 may transmit information on the blood glucose measured by the sensor 110 and the error information of the sensor 110 to the display device 200.

The network usable by the communicator 140 to communicate with the display device 200 is not affected by a specific method. For example, the communicator 140 may use a wireless communication network such as Wi-Fi, Bluetooth, etc. to communicate with the display device 200. For this purpose, the communicator 140 may include a Wi-Fi chip, a Bluetooth chip, a wireless communication chip, or the like.

The display 140 displays various screens. For example, the display 140 may display an error range of the sensor 110 and the blood glucose level of a user. The display 140 may provide a visual feedback for guiding calibration at a time when calibration is necessary.

The display 140 may be implemented as a liquid crystal display (LCD) panel, organic light emitting diodes (OLED), or the like, but is not limited thereto.

The speaker 160 may output various audio. For example, the speaker 160 may output audio directing calibration at the time when calibration is required. The audio directing calibration may be a voice output requiring calibration and also a mechanical sound to notify the calibration timing to a user, or the like.

The haptic provider 170 may generate vibration to a main body of the blood glucose measuring device 100′. To be specific, the haptic provider 170 may provide a haptic feedback to make a user recognize that it is time when calibration is required. The haptic provider 170 may be implemented as a vibration motor, or the like.

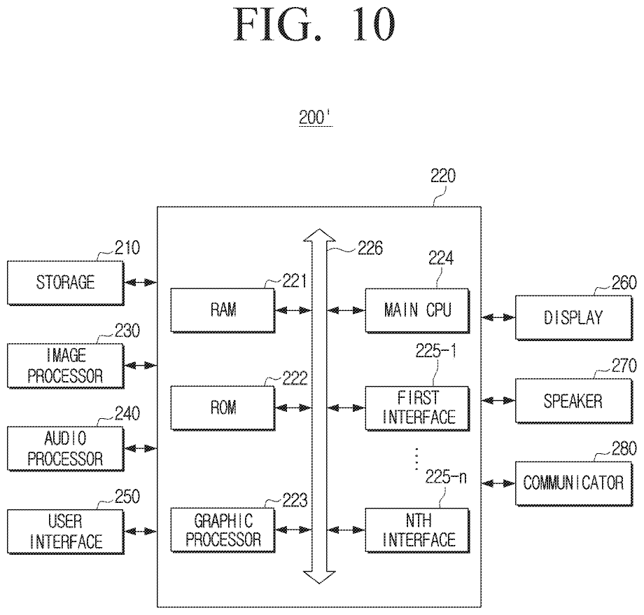

FIG. 10 is a detailed block diagram illustrating a display device according to an embodiment.

Referring to FIG. 10, a display device 200′ according to an embodiment includes a storage 210, a processor 220, an image processor 230, an audio processor 240, a user interface 250, a display 260, a speaker 270, a communicator 280, or the like.

The storage 210 may store an instruction or data related to the components of the display device 200′ and the OS for controlling overall operations of the components of the display device 200′.

Accordingly, the processor 220 may control multiple hardware or software components of the display device 200′ using various instructions or data stored in the storage 210, load instructions or data received from at least one of the other components into the volatile memory, and store the various data in the non-volatile memory.

The processor 220 is configured to control overall operations of the display device 200′.

The processor 230 includes a random access memory (RAM) 221, read-only memory (ROM) 222, the graphic processor 223, a main central processing unit (CPU) 224, first to n^{th} interfaces 225-1 to 225-n, and a bus 226. The RAM 221, ROM 222, graphic processor 223, main CPU 224, first to nth interfaces 225-1 to 225-n, or the like, may be connected to each other through the bus 226.

The first to n^{th} interface 225-1 to 225-n are connected to the various elements described above. One of the interfaces may be a network interface connected to an external device through the network.

The main CPU 224 accesses the storage 210 and performs booting using an operating system (OS) stored in the storage 210, and performs various operations using various programs, contents data, or the like, stored in the storage 210.

The RAM 221 stores an instruction set for booting the system and the like. When the turn-on instruction is input and power is supplied, the main CPU 224 copies the OS stored in the storage 210 to the RAM 221 according to the stored one or more instructions in the ROM 222, and executes the OS to boot the system. When the booting is completed, the CPU 224 copies various application programs stored in the storage 210 to the RAM 221, executes the application program copied to the RAM 221, and performs various operations.

The image processor 230 is configured to perform various image processing decoding, scaling, noise filtering, frame rate conversion, resolution conversion, or the like, for content.

The audio processor 240 is configured to process audio data.

The user interface 250 may receive various user commands. For example, if a user selects a notification UI displayed at the time when calibration of the blood glucose measuring device 100 is necessary, the user interface 250 may receive a calibration command.

The speaker 270 may output various audio.

For example, the speaker 270 may output audio directing calibration at the time when calibration is required. The audio directing calibration may be a voice output requiring calibration and also a mechanical sound to notify the calibration timing to a user, or the like.

The communicator 280 may transmit and receive various data by performing communication with the blood glucose measuring device 100. The communicator 280 may receive blood glucose information from the blood glucose measuring device 100.

A network which the communicator 280 may use for communicating with the blood glucose measuring device 100 is not limited to a specific way. For example, the communicator 280 may use wireless communication network such as Wi-Fi, Bluetooth, or the like. For this purpose, the communicator 280 may include a Wi-Fi chip, a Bluetooth chip, a wireless communication chip, or the like.

The display device 200′ may further include a haptic provider (not shown). The haptic provider (not shown) may generate vibration to the main body of the display device 200′. The haptic provider (not shown) may provide haptic feedback to make the user recognize that it is the time when the calibration is required. The haptic provider (not shown) may be implemented as a vibration motor or the like.

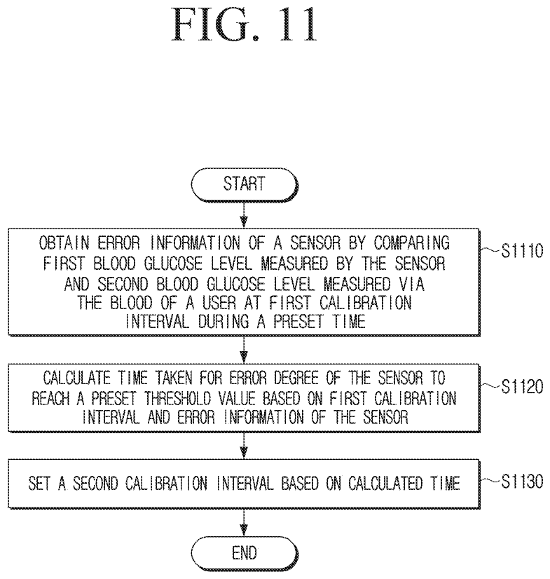

FIG. 11 is a flowchart illustrating an operation method of a blood glucose measuring device according to an embodiment.

According to an embodiment, the blood glucose meter obtains error information of a sensor by comparing a first blood glucose level measured by a sensor with a first calibration period for a preset time and a second blood glucose value measured via the blood of a user in operation S1110. Here, the preset time may be set by the user and set at the time of manufacturing the product.

The blood glucose measuring device calculates a time when the error degree of the sensor reaches a preset threshold value based on the first calibration interval and the error information of the sensor in operation S1120. A preset threshold value may be a value indicative of the accuracy of the sensor. For example, if the preset threshold value is 90%, the time at which the error degree of the sensor reaches a preset threshold value of 90% may refer that the error degree of the sensor is 10%.

The blood glucose measuring device sets the first calibration interval as the second calibration interval based on the calculated time in operation S1130. In the case where the difference between the first and second blood glucose levels is not huge, inconvenience to a user may be minimized by delaying the calibration interval.

The methods according to various embodiments may be implemented as a software or application installable in a related art electronic apparatus.

The methods according to various embodiments as described above may be implemented by software upgrade or hardware upgrade for the conventional electronic device.

The various embodiments as described above may be performed through an embedded server provided in the electronic device or an external server of the electronic device.

A non-transitory computer readable medium storing a program for sequentially performing a controlling method of an electronic apparatus may be provided.

The non-transitory computer readable medium refers to a medium that stores data semi-permanently rather than storing data for a very short time, such as a register, a cache, a memory or etc., and is readable by an apparatus. In detail, the aforementioned various applications or programs may be stored in the non-transitory computer readable medium, for example, a compact disc (CD), a digital versatile disc (DVD), a hard disc, a Blu-ray disc, a universal serial bus (USB), a memory card, a read only memory (ROM), and the like, and may be provided.

The foregoing embodiments and advantages are merely exemplary and are not to be construed as limiting the disclosure. The present teaching may be readily applied to other types of devices. Also, the description of the embodiments of the disclosure is intended to be illustrative, and not to limit the scope of the claims, and many alternatives, modifications, and variations will be apparent to those skilled in the art.

While various embodiments have been illustrated and described with reference to certain drawings, the disclosure is not limited to specific embodiments or the drawings, and it will be understood by those of ordinary skill in the art that various changes in form and details may be made therein without departing from the spirit and scope as defined, for example, by the following claims and their equivalents.

What is claimed is:

1. A blood glucose measuring device comprising:
a sensor configured to measure blood glucose via a body fluid of a user; and
a processor configured to:
obtain error information of the sensor by comparing a first blood glucose level measured by the sensor, and a second blood glucose level measured via the blood of the user, at a first calibration interval during a preset time,
calculate a time taken for the error range of the sensor to reach a preset threshold value, based on the first calibration interval and the error information of the sensor, and
set the first calibration interval as a second calibration interval based on the calculated time.

2. The blood glucose measuring device of claim 1, wherein the processor is further configured to:
obtain first error information of the sensor by comparing a first blood glucose level measured by the sensor and a second blood glucose level measured via the blood of the user, at the first calibration time included in the preset time,
obtain second error information of the sensor by comparing a first blood glucose level measured by the sensor and a second blood glucose level measured via the blood of the user at a second calibration time in which a time corresponding to the first calibration interval has passed from the first calibration time, and
obtain error information of the sensor based on the first error information and the second error information.

3. The blood glucose measuring device of claim 2, wherein the processor is further configured to obtain error information of the sensor by further considering a physical error range of the blood glucose measuring device included in each of the first error information and the second error information.

4. The blood glucose measuring device of claim 2, wherein the processor is further configured to:
based on a time for the error range of the sensor to reach a preset threshold value being shorter than the first calibration interval, set the time to reach the preset threshold value as the second calibration interval, and
based on the time to reach the preset threshold value being longer than the first calibration interval, and based on the time to reach the preset threshold value being in a preset correlation with the first calibration interval, set the first calibration interval as the second calibration interval.

5. The blood glucose measuring device of claim 4, wherein the processor is further configured to:
based on the time to reach the preset threshold value being longer than the first calibration internal and shorter than two times of the first calibration interval, set the second calibration interval to be identical with the first calibration interval, and
based on the time to reach the preset threshold value being longer than the two times of the first calibration interval and shorter than three times of the first calibration interval, set the second calibration interval as two times of the first calibration interval.

6. The blood glucose measuring device of claim 2, wherein the processor is further configured to:
based on an error of the sensor being calibrated at the first calibration time, generate information on a blood glucose level including a blood glucose level measured by the sensor in a preset time interval unit from the first calibration time and a predicted error range of the sensor based on the error information of the sensor at the blood glucose measurement time.

7. The blood glucose measuring device of claim 1, wherein the processor is further configured to provide an alarm to direct a user to calibrate an error of the sensor at a time corresponding to the second calibration interval.

8. A blood glucose measuring system including a blood glucose measuring device and a display device, the system comprising:
the blood glucose measuring device configured to:
obtain error information of a sensor of the blood measuring device by comparing a first blood glucose level measured via a body fluid of a user and a second blood glucose level measured via the blood of the user, at a first calibration interval during a preset time, based on the error of the sensor being calibrated at a first calibration time,
measure a blood glucose level in a preset time interval unit from the first calibration time,
predict an error range of a sensor based on the error information of the sensor at the measurement time of the blood glucose,
transmit, to the display device, information on a blood glucose level including the measured blood glucose level and the predicted error range of the sensor at the measurement time, and
a display device configured to receive and display blood glucose information from the blood glucose measuring device.

9. The blood glucose measuring system of claim 8, wherein the blood glucose measuring device is further configured to:
based on the first calibration interval and the error information of the sensor, calculate a time taken for the error range of the sensor to reach a preset threshold value on the basis of the first calibration interval and the error information of the sensor,
set the first calibration interval as a second calibration interval on the basis of the calculated time, and
transmit information on the second calibration interval to the display device,
wherein the display device is further configured to provide a user interface (UI) for guiding to calibrate an error of the sensor based on information on the second calibration interval.

10. The blood glucose system of claim 9, wherein the display device is further configured to, based on the predicted error range of the sensor at the measurement time corresponding to a preset threshold value, provide a preset visual feedback.

11. The blood glucose system of claim 8, wherein the display device is further configured to, based on a change amount of blood glucose measured by the sensor exceeding a preset threshold value, provide the UI by reflecting additional error information based on the change amount of blood glucose to the predicted error range of the sensor at the measurement time.

12. A method for measuring blood glucose using a blood glucose measuring device, the method comprising:
obtaining error information of a sensor of the blood measuring device by comparing a first blood glucose level measured via a body fluid of a user and a second blood glucose level measured via the blood of the user, at a first calibration interval during a preset time;
calculating a time taken for the error range of the sensor to reach a preset threshold value, based on the first calibration interval and the error information of the sensor; and
setting the first calibration interval as a second calibration interval based on the calculated time.

13. The method of claim 12, wherein the obtaining the error information comprises:
obtaining first error information of the sensor by comparing a first blood glucose level measured by the sensor and a second blood glucose level measured via the blood of the user, at a first calibration time included in the preset time, to calibrate the error of the sensor;
obtaining second error information of the sensor by comparing a first blood glucose level measured by the sensor and a second blood glucose level measured via the blood of the user at a second calibration time in which a time corresponding to the first calibration interval has passed from the first calibration time; and
obtaining error information of the sensor based on the first error information and the second error information.

14. The method of claim 13, wherein the obtaining the error information comprises obtaining error information of the sensor by further considering a physical error range of the blood glucose measuring device included in each of the first error information and the second error information.

15. The method of claim 13, wherein the setting the second calibration interval comprises:
based on a time taken for the error range of the sensor to reach a preset threshold value being shorter than the first calibration interval, setting the time to reach the preset threshold value as the second calibration interval; and
based on the time to reach the preset threshold value being longer than the first calibration interval, and based on the time to reach the preset threshold value being in a preset correlation with the first calibration interval, setting the first calibration interval as the second calibration interval.
